(19) United States
(12) Patent Application Publication
(10) Pub. No.: US 2003/0088365 A1
(43) Pub. Date: May 8, 2003

(54) System and method of drug development for selective drug use with individual, treatment responsive patients, and applications of the method in medical care

(76) Inventor: Robert Becker, Carrabassett Valley, ME (US)

Correspondence Address: CUMMINGS AND LOCKWOOD, GRANITE SQUARE, 700 STATE STREET, P O BOX 1960, NEW HAVEN, CT, 06509-1960, US

(21) Appl. No.: 10/182,785

(22) PCT Filed: Oct. 26, 2001
(86) PCT No.: PCT/US01/49457

(51) Int. Cl. G06F 19/00; G01N 33/48; G01N 33/50

(52) U.S. Cl. 702/19

(57) ABSTRACT

A system and method is provided for medical researchers into drug procedure or intervention, or device efficacy, safety, economics or use, for developing and testing a decision model that determines for patients individually the probable efficacy, safety, economic benefits and use of drugs or medical devices. The model uses studies to determine the reliability of measurements, criteria of clinical significance, criteria of statistical significance, studies of the internal validity of patient assessments under both double-blind placebo controlled and non-double-blind non-placebo controlled conditions, methods of confirming the predictions from the clinical trial model, and studies of long-term predictions of health status from outcome measurements, and clinical trial or other medical research designs, to identify each individual's response to treatment. The decision model improves the implementation of scientific and medical standards of patient care in medical practice and is applicable in medical care, drug development and regulation, health care financing, electronic medical records, and pharmacy practice.

### CROSS-REFERENCE TO RELATED PRIORITY APPLICATIONS

This patent application claims priority on the present inventor's following co-pending provisional patent applications which are each hereby expressly incorporated by reference as part of the present disclosure: serial No. 60/258,262, filed Dec. 26, 2000, entitled “Method of Administering ChEIs for treating Alzheimer's Disease”; serial No. 60/274,981, filed Mar. 12, 2001, entitled “Method of Drug Development for Selective Use with Individual, Treatment Responsive, Patients,” and serial No. 60/301,526, filed Jun. 28, 2001, entitled “Method of Drug Development for Selective Use with Individual, Treatment Responsive, Patients and the Applications of the Method of Drug Development in Medical Care”.

### FIELD AND OVERVIEW OF THE INVENTION

The present invention relates to systems and methods that use randomized assigned subjects in double-blind, placebo-controlled, clinical trials (“CTs”) or other medical research designs to evidence support for strong hypotheses that predict the efficacy and safety of drugs, medical interventions, procedures, treatments or devices (hereafter collectively referred to as “treatments” or “drugs”) or other treatments in individual patients. The present invention differs from current practice in that current CT and clinical research evidence support for weak hypotheses by determining the efficacy and safety of a treatment for a group of treated patients. CT or research extended with the methods of the invention, on the other hand, evidence support for strong hypotheses by determining efficacy and safety of a treatment selectively among individual patients.

### BACKGROUND OF THE INVENTION

Modern drug and medical device development depends on CTs to demonstrate that a group of patients treated with a drug show statistically significant, clinically desired differences from an untreated group of patients. (Senn, 1977; Sacristan et al., 1998). The statistically grounded inferences of efficacy and safety of a drug or device depend upon there being no systematic differences, other than the treatment (the drug(s) or device(s)) being tested between the treated and untreated groups in the CT. To allow this assumption, medical and scientific investigators use as conditions of CTs a randomly assigned convenience sample of a target population, treated with both the subjects and investigators blind to the assignment of subjects to a drug treatment or placebo treatment group. The modern CT allows the investigator to evidence support for a weak alternative hypothesis that the drug is more effective than placebo by rejecting on probability grounds a weak null hypothesis of no group differences. (Senn, 1997, 49-51). However, evidence that a treatment is effective and safe in a group of patients does not provide the practicing physician with grounds to conclude that the drug or device is effective or safe in his or her individual patient. The clinician must use unsystematic clinical experience and judgment not-validated scientifically (Guyett et al., 2000) to estimate the importance of group clinical trial evidence—evidence about “an average randomized patient” (Feinstein and Horwitz, 1998)—for her individuial patient. The clinician does not have scientific validations that the CT average randomized patient resembles her individual patient in all ways important to successful treatment; that her clinical methods of assessment have sufficient reliability and validity (e.g., are sufficiently free from random or systematic error from one administration to another and express the actual condition of the patient or the true clinical course of the patient) to be grounds for clinical judgments about the patient's response to the therapeutic intervention; that the criteria of clinical significance she selects reflect the current standard of medical knowledge; in decision making that the odds for efficacy and safety for the mean patient in the group apply to her individual patient; and so forth. The evidence currently available from clinical trials has been severely criticized for being insensitive to “clinical nuances” that are crucial considerations in patient care. (Feinstein and Honwitz, 1998).

In contrast to these limitations in current clinical trial practice, it would be desirable to develop a system and method that use CT research:

I. To implement as the aims of CT investigation to evidence treatment effects reliably and validly in individual patients.

II. To implement these aims that:

A. Establish reliability of measurement.

To establish the conditions of use of an outcome measure such that the error of measurement will not interfere with the intended clinical uses of the outcome measure in medical decision making.

B. Set criteria of clinical significance.

Use criteria of clinical significance derived from the current state of medical knowledge and standards of care to identify individual patients as responders or not responders to a treatment.

C. Set criteria of statistical significance.

Use criteria of statistical significance to judge inferences in the clinical trial and in patient care to implement the inference to an individual being a responder or not; to establish the probability that an individual's clinical course could occur under placebo or under treatment conditions; to distinguish as different two clinical courses; to set confidence intervals; and so forth.

D. Select the reference that determines the probability of a treated patient's response in one treatment arm being dependent on the treatment in that condition.

E. Defend the internal validity of both the double-blind, placebo-controlled and the post-double-blind, placebo-controlled open periods of the CT.

F. Confirm as needed the status of individual patients, especially those with a low probability of meeting criteria of clinical and statistical significance.

G. Support the predictions of long-term health outcomes from the surrogate variables that describe the patient's clinical course.

H. Methods of analysis.

Draw on statistical, clinical trial, medical and other methods of analysis as needed to implement the system and method.

III. To apply the evidence from extended clinical trials:

A. In patient care;

B. In drug development and regulation;

C. In health care financing and formulary maintenance;

D. In electronic medical records; and/or

E. In pharmacy practice.

Becker and Markwell (2000) discuss some of the limitations in current CT research. The errors in the tests used to assess clinical status of Alzheimer's disease (“AD”) patients is sufficiently large to obscure the drug effects leaving the practicing physician with neither research CT derived grounds, nor reliable clinical assessments of individual patients to inform clinical judgments of treatment management. For the great majority of AD patients, clinical assessments are unreliable indicators of patient status. AD treatment illustrates an extreme of the failing of modern CT methods to inform individual medical decisions. On the other end of the spectrum, even medicine's most reliable assessments—for example, laboratory examination—offer an interpretation based on a normal range of test results which allow 5% (or thereabouts) of all routine observations to be classified as outside the normal range. Current CT methodology does not develop a model that takes account of this variable error range among outcome measures to provide to the practicing physician both evidence for the efficacy and safety of a treatment, and a model that takes into account the error in methods of assessment to reliably assess the effects of the treatment in an individual patient. With laboratory tests widely used, 5% of all uses give evidence that can be interpreted as abnormal or pathological. As a result, patients are subjected to the risks of more examinations or to unnecessary treatments for non-existent disease. Accordingly, it would be desirable to provide a system and method that address these limitations.

The present inventor is not aware of the study in modern CTs of the error variance in the use of clinical examination methods and laboratory procedures. For example, CTs do not characterize individually the different courses taken by different patients when they compare groups in treatment arms. Statistical significance for the presence of a difference among groups is not evidence of clinical significance of a treatment for individual patients. (Hall, 1993; Pledger, 1993; Borenstein, 1994; Senn, 1997, p. 115-117; Becker and Markwell, 2000). Even more detailed than customary analyses of CTs or research—determinations of effect size, demonstrations of homogeneity or heterogeneity of response, plots to determine false positive and false negative relations, taking into account test-retest reliability of outcome measures, and so forth—in current practice do not provide a model the clinician can apply to assess the response of an individual patient and to predict and test the patient's future course. (Becker and Markwell, 2000). The modern CT methods force the clinician to generalize from group outcomes to the individual course of his or her patient. (Siegel, 1956, p.2; Hays. 1963, p. 250, 296-299; Senn, 1977, p. 28; Davis, 1994). Accordingly, it would be desirable to enable the medical investigator to conduct and interpret CTs such that clinicians could incorporate into patient care with established reliability knowledge of individual patient experiences in a CT.

Clinicians need to confirm their estimations of status for a patient. The n-of-1 trial provides one resource when randomized controlled trials do “not help in deciding treatment for an individual patient.” (Drug Ther Bull, 1998). Accordingly, it would be desirable to make the n-of-1 trial—currently the only clinical research method believed to be valued more highly than the clinical trial—more practical and effective. (Guyatt et al., 2000).

Another problem in CT application in patient care arises because clinical trials fail to test the efficacy of drugs over the full period of their use in patients. Typically, medical treatments are used in clinical practice over a lonaer period than the duration of the clinical trial. (Vickers and de Crean 2000;DeDeyn and D'Hooge 1996). In addition, n-of-1 trials may be needed to determine if a treatment is benefiting a patient in some circumstances. (Guyatt et al., 1990; Larson et al., 1993; Backman and Harris, 1999). However, the n-of-1 trial has limitations: the randomization procedure is time consuming; the trial exposes the patient to periods of no treatment in placebo treatment; and the trial often has less statistical power than a CT, increasing the likelihood of erroneously continuing or discontinuing a treatment on the basis of the n-of-1 trial results, or the results being inconclusive. Therefore, the clinician frequently will not want to use the n-of-1 trial technique when its use can be avoided. (Johannessen and Fosstveldt, 1991). Accordingly, it would be desirable to develop a model derived from the CT, that is tested in both the blind and open phases of the CT, and that overcomes at least some of the limitations making n-of-1 trials more practical.

Evidence-based medicine currently embodies the modern standards for patient care. Evidence-based medicine (“EBM”) “acknowledges that intuition, unsystematic clinical experience, and pathophysiologic rationale are insufficient grounds for clinical decision making, and stresses the examination of evidence from clinical research.” (Guyatt et al., 2000, p. 1291). In the application of CTs to therapeutic decision making, it would be desirable to replace “unsystematic clinical experience” with probabilities supporting evidence of individual patient responses from CTs. CTs, or “randomized trials” as they are sometimes called, are one of the highest standards in clinical research, in practical research, and in regulatory perspective constitute the state of the art. (Guyatt et al., 2000, p. 1292). Modern scientific medicine depends upon the quality of the research evidence. The practitioner of EBM “must be able . . . to critically appraise the research evidence; and to apply that evidence to patient care.” (Guyatt et al., 2000, p. 1291). The goal of modern medicine is to base patient care decisions on the highest quality scientific evidence. (Guyatt et al., 2000; Ellis et al., 1995). Patient care decisions require scientific grounds for the generalized safety and efficacy of the proposed treatment and scientific grounds showing that the treatment is safe and effective in the individual patient under treatment. Current methods of drug development cannot fully meet these standards of scientific medicine. Because of inadequate and inappropriate design and analysis of CTs and presentation of research results to meet the demands of practice, the above-described CT methods do not allow a critical appraisal of the research evidence implications for individual patients, and do not provide scientific grounds for using the CT results to reach care decisions with individual patients without calling on unsystematic clinical experience. As a result, the prior art methods of CTs can mislead the clinician in applying the scientific medical evidence of treatment efficacy to patient care.

Because they require personal clinical judgments for interpretation of patient care implications, modern CT methods are open to erroneous interpretations. A representative range of errors in interpretation that can result from modern CT methodology occur in the recent development of AChEIs and ChEIs for use in AD. The errors are evidenced in clinical trial reports as demonstrated by Becker and Markwell (2000) and in Raskind et al. (2000), in clinical reports such as Rogers and Friedhoff (1998), Matthews et al., (2000), Shua-Haim et al. (2000), in expert reviews such as Giacobini and Michel (1998), and in FDA-approved professional and public advertising for drugs of this class where drugs of the classes AChEI and ChEI are claimed to improve patient performance in AD and are recommended for prescription based on this claim of efficacy. Such drugs are presented as improving the cognitive performance of patients when the data reveal that they better sustain the test performance than placebo, as reported by Becker et al. (1996, 1998) and discussed in Becker and Markwell (2000). The drugs are represented as more effective than justified by the variance in outcomes in the placebo groups. (Becker and Markwell, 2000). However, the authoritative reports and recommendations of experts do not address the inability of the physician to reach reliable and valid assessments of the individual patient, the effect of error in test-retest on outcome measures, and the problems these assessment deficiencies raise for scientifically and medically sound medical decision making. (Becker and Markwell 2000). The current methods of reporting do not take into account the inability of clinicians to assess individual patient responses. In addition, the lack of recognition of this failing in current clinical methods leads to ungrounded decisions about dosing changes and clinical response. (Becker and Markwell, 2000). As the citations in this paragraph witness, clinicians are encouraged to assess the benefits from drug administration and to reach clinical judgments about dosing and management using scientifically inadequate analyses of CTs and unreliable and invalid clinical assessments. (Becker and Markwell 2000).

Cox (1958), Hays (1963), Senn (1997) and other authorities in experimental design and statistics encourage a weak and abstract end in medical research—“in practice the experimenter always acts as though he is deciding between two hypotheses” (Hays, 1963, p. 247)—rather than the development of a scientifically proven model or tested method of application to individual patients. It would, therefore., be desirable to provide a system and method pursuant to which hypothesis testing addresses strong hypotheses, i.e., hypotheses about the individuals, not the groups in the research. Weak (group) hypotheses and hypothesis testing should be at best, intermediate to the aim of the research to provide a more useful, directly applicable, individualized model of efficacy, or safety, or use of a treatment.

### SUMMARY OF THE INVENTION

In the system and method of the present invention, the statistical rejection of a strong null hypothesis in a CT opens to consideration a strong research hypothesis of treatment efficacy conditional on the specific conditions of the application of the drug or device to individual patients and the individual responses of patients to the treatment. The system and method of the invention uses CTs to develop, test and demonstrate the use of a model decision rule or rules that can then be applied in medical practice to individual patients to reliably assess probable drug efficacy or lack of efficacy in each individual patient on an ongoing basis and to predict the probable ultimate outcome from continued treatment. The system and method of the invention preferably facilitates the ongoing assessment of patients by n-of-1 studies.

Accordingly, a principal aim of the present invention is to provide the medical practitioner scientific and statistical evidence of individual patient responses to treatment. The system and method of the invention develops and tests a model that assesses individual patient responses to drugs. The model generated by the invention in the clinical trial provides physicians in clinical practice with a scientifically and statistically founded means to determine whether an individual patient currently benefits or not from the treatment, and the most probable outcome for each patient as an individual (i.e., immediate or ultimate clinical benefit, or treatment failure) with continued treatment of the individual patient. In current practice, on the other hand, physicians apply clinical trial evidence about drug effects in groups of patients to an individual patient using unsystematized clinical experiences and clinical judgments of the patient's responses to treatment as the grounds of individual patient medical care decisions. Thus, a significant advantage of the present invention is that it may be applied in clinical drug development to provide the practicing physician with the needed, but currently unavailable resources, to apply CT evidence to individualized patient care by drawing on a research tested model that evaluates individual patient responses.

In a currently preferred embodiment of the present invention used in the research and development of drugs or medical devices, the system and method generates and tests a model of individual patient assessment of drug or device effects that has a wide range of applications:

A. In patient care: to improve the pharmacological evidence, particularly evidence that can improve the decision making for an individual patient, available to the practicing physician who must evaluate the efficacy and safety of a drug or medical device in each individual patient;

B. In drug development:

1) In investigational new drug (“IND”) research supporting an initial new drug application (“NDA”) approval by the Food and Drug Administration of the United States (“FDA”) or a comparable regulatory review and approval by the agencies of other countries (unless otherwise indicated, “NDA” is used herein to refer to any form of regulatory approval, and “drug” is used herein to refer to a device, intervention, procedure, or other treatment);

2) In research supporting a subsequent IND and NDA to make more selective the clinical applications of the drug in patient care; and

3) For a treatment too expensive in use to justify its development without the method of the invention;

C. In health care financing and formulary maintenance: As a resource in cost-benefit or other evaluative decisions reached by insurers or funders of medical care pharmacy committees of hospitals or other health care organizations, or other groups that control drug availability to physicians and patients; and for selective use of more economical methods of treatment in those patients who show no additional benefits from more expensive methods of treatment;

D. In electronic medical records:

1) Integrated into electronic medical record systems; and

2) To provide a clinical trial, evidence-based assessment of personal response to a prescribed treatment accessible to each individual patient; and

E. In pharmacy practice: To specify the conditions for controlled dispensing of the prescribed drug for maximizing the efficacy and safety of use in patients.

Other objects and advantages of the present invention will become readily apparent in view of the following detailed description of preferred embodiments and accompanying drawing.

### BRIEF DESCRIPTION OF THE DRAWINGS

FIG. 1 is a graphical illustration of a statistical model generated by a CT in accordance with a preferred embodiment of the present invention.

### DETAILED DESCRIPTION OF THE PREFERRED EMBODIMENTS

I. The Present Invention Aims to Evidence Treatment Effects in Each Individual Patient in a CT Reliably and Validly.

The present invention is directed to a system and method of CT, CT design, CT analysis, and CT application, or any other research, to determine the efficacy, safety, economic benefits or costs, or uses of a drug, intervention, procedure, or medical device. It is assumed that CTs or other evaluative research that use the method of the invention incorporate current standards for CT research, including but not limited to randomized assignment of subjects to treatment arms, unbiased selection of the sample from the population eligible for the applications, double-blinding, placebo-controls, and so forth. The present invention adds to current CT practice the following methods that extend CTs to identify treatment effects in individuals:

II. Implementing the Aims with the Present Invention.

A. Establish Reliability of Measurement and an Assessment Plan for the CT.

An investigator studies the test-retest reliability of outcome measures prior to the CT by repeated administrations of candidate measures to a patient sample. The errors of measurement in comparisons of single applications, or of descriptive statistics summarizing multiple applications, of an outcome measure, provide grounds to plan the trial; to select sufficiently reliable outcome measures to meet the aims of the study; to develop an assessment plan for using repeated measures of outcome in the clinical trial; to choose a scalar summary statistic to express each patient's clinical course (Frison and Pocock, 1997; Becker and Markwell, 2000; Senn et al., 2000; Weinberg and Lagakos, 2001); and to calculate confidence intervals of measurement for the outcome measures using established methods. (Becker and Markwell, 2000). The aim of the pre-trial test-retest reliability studies and assessment planning is to estimate reliably the patient's true clinical course by controlling for the uncertainties introduced by measurement error or other sources of variance. To identify individual patient responses, repeated measures must provide sufficient reliability for the scalar summary statistic to accurately express the true clinical course of each individual in the trial. In analysis of individual patient courses, to express effects from error of measurement, scalar summary statistics are bounded by the appropriate confidence interval of measurement.

Analysis of co-variance or other statistical adjustments may be required in a manner known to those of ordinary skill in the pertinent art to take into account baseline, pre-treatment, or within trial effects of independent variable differences among patients or within trial effects. (Hays, 1963, p. 564; Senn, 1997, pp. 95-104). The scalar summary statistics for each patient are fitted to a statistical or other model, such that the changes in status over time for each patient can be analyzed. The method of the invention can be used in regression on baseline status variables to predict treatment effects in individual patients using methods of regression in a manner known to those of ordinary skill in the pertinent art.

When a single administration of an outcome measure compared to another single administration error of measurement affects adversely the accuracy of measurement of the patient's course required by the aims of the study, multiple assessments are used to provide the data points for the summary scalar statistic of the patient's course. The assessment plan derived from the pre-trial reliability study specifies study design and analysis—how frequently outcome measures are administered to patients, how multiple administrations avoid carryover effects, and so forth, as required in assessment and measurement, and whether a descriptive statistic summarizing multiple administrations or a single occasion of administration are used in data analysis to test the hypotheses of the study and to develop the model for clinical treatment that will express the efficacy, safety, uses, or other dimensions of the treatment important to individualized patient care decisions. Outcome measure reliability can be re-studied at any time during or after a CT to establish an assessment plan that meets the aims of the study or aims of a physician's application in patient care.

B. Set Criteria of Clinical Significance.

Criteria of clinical significance are derived from the current state of medical knowledge and standards of care. These criteria identify individual patients as responders or not responders to a treatment. Criteria of clinical significance designate the scalar summary statistic that summarizes each patient's clinical course as a clinically significant response or non-response to the treatment received. Since the patient's true course is defined as occurring within the confidence intervals of measurement, and a criteria of clinical significance may be defined by medical science as a confidence interval of measurement, the judgment of response or non-response can be categorical or probability based according to the conditions set by the investigators, the aims or design of the study, or current medical, statistical, or scientific standards of practice.

C. Set Criteria of Statistical Significance.

The criteria of statistical significance judge inferences in the clinical trial. It is customary in CT to set a criteria of p=0.05, or one chance in 20 that an outcome can occur by chance, as the criteria of statistical significance to judge group outcomes among treatment arms. This, or other scientifically, medically, or statistically, justifiable criteria are used in the extended CT to implement the inference to an individual being a responder or not; to establish the probability that an individual's clinical course could occur under placebo or under treatment conditions; to statistically support the internal validity of a study; to select confidence intervals; to distinguish as different two clinical courses; and so forth. The criteria conditions governing specific comparisons often will be specified in relation to the aims or design requirements of the study because, as discussed under D. below, the probabilities associated with the use of confidence intervals in judging the difference between two estimates are sensitive to the statistical characteristics of the estimates. (Schenker and Gentleman, 2001). Also, risk-benefit ratios will apply in particular instances in a manner known to those of ordinary skill in the pertinent art based on the teachings herein. For example, a relatively high probability that a specific patient's response after receiving a treatment could occur under placebo conditions in the CT can lead to different patient care decisions under different conditions. Where the risk of continued treatment is low, or the risks of discontinuing treatment if it is effective could be extremely serious for the patient, the physician may choose to continue treatment, in effect accepting as the criteria of statistical significance more than one chance in 20 of the response not being an effect of treatment. Researchers and clinicians will accept different probabilities, different confidence intervals, and so forth, according to customary scientific, statistical, decision making, and medical practices, and because they will balance risks and benefits in taking decisions or in inferences.

D. Select the Reference that Determines the Probability of a Treated Patient's Response in One Treatment Condition being Dependent on the Treatment in that Condition.

After characterizing an individual under the criteria of clinical significance, the investigator and treating physician ask for a probability that a patient's response is a consequence of treatment. The statistical probability of a patient's response occurring as an effect of treatment can be estimated in various comparisons: the probability that the actively treated patient's course would occur under comparison or placebo conditions; whether the confidence interval of the actively treated patient's course overlaps the mean comparison, or the mean placebo treated patient course, or its confidence intervals; as an odds ratio of the cumulative frequency of the patient's course among actively treated patients divided by the cumulative frequency among comparison or placebo treated patients; as an exact probability determined by a randomization test; and so forth. The conditions of comparison of the individual patient's course to the patient courses in the clinical trial are aim and design specific, both because differences in aims and design require different comparisons, and because the statistical characteristics of the estimates, point, descriptive, summary scalar, with different variances, confidence intervals, and so forth, interact with how conservative an estimate need be in the selection of the statistical test.

The method of the invention is useful when a placebo group cannot be justified as a CT comparison to the treatment of interest. If a treatment is shown effective and safe for a disease with serious consequences if left untreated, the disease is progressive, and so forth, the availability of a treatment can preclude the use of placebo treatments in future CT. Under the method of the invention, the comparison of efficacy is not to placebo, but to criteria of clinical and statistical significance using outcome measures of demonstrated reliability to provide confidence intervals for individual patient's clinical courses. Secondary support for treatment efficacy comes from the distributions of responders among treatment arms refining the equivalence CT where two active treatments are compared. Two treatments may appear equivalent, one not superior to the other because the confidence intervals for the means overlap. Nonetheless, individual patient courses may show that for some patients one treatment is superior to the other and with clinical advantages not evidenced in the group comparisons.

The aim of the extended clinical trial of the invention is not to evidence for the pharmacological scientist that a drug is different from placebo in the human species or in a subset of the species. Rather, the aims are to develop a decision model useful to the clinician deciding whether each patient shows an optimal clinical response and if the response is an effect of treatment. One implementation of these aims may be to calculate an odds ratio for each clinical course occurring under treatment and placebo conditions. These odds ratios (for example, probability that the surrogate outcome indicates a treatment effect or will result in a long-term health benefit—(T)/{1−p(T)}/p(P)/{1−p(P)} (see G below for discussion of surrogate variables)) can improve the precision of estimation by a practitioner of true-positive and false-negative ratios for a patient in a clinical decision model. Thus, the design of the CT takes into account the specific applications of the CT to best support the clinician in patient care.

E. Determine the Internal Validity of Both the Double-blind, Placebo-controlled and the Post-double-blind, Placebo-controlled Open Periods of the CT.

In current CT practice, the presence of a difference under double-blind conditions between groups at a level of chance sufficient to reject the null hypothesis lays the grounds for the consideration of the alternative or research hypothesis. Since the research samples are convenience, not randomly chosen, a permutation or randomization test is preferred to evidence statistically internal validity. The same approach to statistical testing for internal validity can be used in the extended clinical trial. The extended clinical trial also can be tested statistically for internal validity by first applying criteria of clinical significance and then using permutation or randomization tests to demonstrate the probabilities of occurrence of outcomes bearing on the strong null hypothesis. The statistical testing of a distribution of patient outcomes among different levels of treatment, different treatments, or a treatment and placebo can be combined with Generalizability and Decision studies of individual patients, individual patients grouped by confidence intervals (for example, where a long-term health outcome is an aggregate derived from the surrogate outcomes for all patients with courses that fall within the confidence interval calculated for the patient of interest in the research or to a practitioner) categories of patients (such as those with outcomes that fall above and below clinical criteria of response or in relation to different Aggregated responses and confidence intervals developed out of the aims or design of the study).

Extended clinical trials can also provide statistical test support for internal validity of the open or non-blind phase of the CT. Generalizability and decision studies extend the conclusions of efficacy and safety beyond the period of the CT or research; limit the internal validity of extension by follow-up evaluations of patients in the original research (when changes in the distributions of outcomes no longer statistically differ significantly the follow-up data are no longer internally valid as additions to the analysis of efficacy and safety); and to further extend the generalized conclusions of efficacy and safety by n-of-1 trials, or randomized assignment to a new CT of drug and placebo, carried out on patients in the original research after prolonged exposure to the drug (see F. below). This extension calls on the same assumptions of external validity required to apply any clinical trial beyond the period of double-blind comparison. The currently preferred embodiment of the present invention requires first, a reliable projected estimation of placebo treated patient outcomes after the double-blind as a comparison group to establish a probability for the treated patient's status and second, evidence against bias influencing the open assessments of patients in followup. Evidence from test-retest reliability studies comparing blind and open use of outcome measures, evidence demonstrating no significant effect (no effect greater than the confidence interval of measurement) on patient courses following the transition from blind to open treatment conditions, and so forth, can be generated in the CT to support the reliability of outcome measures in the hands of practitioners who establish test-retest reliability and follow the assessment plan required by the aims of treatment. Even if the CT in open extension loses internal validity, continued compliance with the reliability of measurement and criteria governing the double-blind model can support the validity of the model to indicate response and treatment dependence of response if other assumptions are met.

F. Confirm (as Needed) the Status of Individual Patients, Especially Those with a Low Probability of Meeting Criteria of Clinical and Statistical Significance.

Extended clinical trial methods of the invention make n-of-1 trials more practical as methods to confirm individuals as meeting criteria or experiencing a clinically significant or statistically significant effect of treatment compared with placebo. In an n-of-1 trial, any patient can be randomized to a sequence of double-blind, placebo and drug treatments and the scalar summary statistics for the periods of treatment compared to develop probabilities for and against a significant drug effect in the patient. With the modified clinical trial model, the number of sequences can be abbreviated because the interpretations do not depend on only the within n-of-1 trial data. In an extended trial model, interpretations of scalar statistics summarizing the clinical courses during active and placebo treatment draw on the original criteria for clinical and statistical significance, the confidence intervals of measurement, and the distributions of outcomes among treatment arms. In relation to the aims or design of the trial or the clinical application of the model from the trial, the criteria of clinical significance as an absolute measure of an active treatment course or as interpreted as an incremental difference from the placebo course of the patient can be chosen to judge the efficacy demonstrated by an individual in an n-of-1 trial. Statistical significance can meet the criteria or be relaxed, or tightened, under the considerations raised in C. above. One convenient test is provided by whether the patient both maintains the open treatment course under blind active treatment and deviates significantly under blind placebo treatment during the n-of-1 trial. The confidence intervals of measurement determined in the CT indicate change; a placebo course that crosses the 90% confidence interval of measurement of the active treatment course evidences less than 5% chance occurrence of the difference. These resources from the extended clinical trial model make n-of-1 trials more practical. Currently n-of-1 trials suffer from requiring a number of sequences of placebo and drug and from low power in a statistical analysis. The method of the invention overcomes these difficulties by allowing one blind, randomly assigned, placebo comparison to drug to evidence efficacy or the lack, or safety or the lack, because of the power added by reliability, generalizability theory, randomization tests, producing confidence intervals of measurement and within subject comparisons. Of course, the usual considerations must be addressed to exclude compromise of external validity.

G. Support the Individualizing of Predictions of Long-term Health Outcomes from Surrogate Variables.

Often CT outcome measures are surrogates for clinically important health outcomes: blood pressure for stroke, heart disease, kidney failure; blood glucose for blindness, kidney failure, cardiovascular disease, and so forth. The method of the invention, by characterizing individual courses, can generate probabilities for long-term health outcomes specific to distinct clinical responses—individual courses; course intervals bounded by confidence intervals of measurement, and so forth. The method of the invention thus more precisely equates differences among courses measured by surrogate outcome variables with different predicted long-term health effects. This can improve risk-benefit evaluations of treatment decisions.

H. Methods of Analysis.

Extended clinical trial analysis draws on statistical, clinical trial, medical and other methods of analysis as needed to implement the methods of the invention. Regression, exploratory data analysis, generalizability, descriptive statistics, analysis of variance and co-variance, permutation and randomization testing, and so forth have already been described and the methods referenced to the existing art.

The method of the invention to use the individual patient as the unit of analysis, may require as an item of data for an individual patient any of the following:

1) A medical assessment at an instant in time, for example, a blood pressure, a laboratory test result, a score or single response to a question or other stimulus, or any other result from a medical examination.

2) An aggregated score or response where established methods provide a questionnaire or rating scale score, a summary score or quantification of a laboratory, or imaging or other medical study of the patient.

3) A profile of the patient over time, for example, a defined time period, of an hour, day, week, or other period of time, where an aggregated measure(s) over the time become the unit of repeated measurements, comparison, and analysis.

4) Any other indicator used to assess treatment efficacy or health status in medical practice or research.

In addition to the methods of analysis already indicated, the methods of this invention, as needed to reach the aims of the invention and study in which the methods are used, can employ exploratory data analysis and methods of analysis known to anyone familiar with the art of drug development and statistical analysis of drug research, including but not limited to:

1) Linear regression methods for determining the slope of a patient's responses on repeated measures over time (Hayes, 1963, pp. 490-499);

2) Means, medians, standard deviations, and other descriptive statistics to express or aggregate for each individual patient the non-progressive changes after an initial treatment effect (Hayes, 1963, pp. 161-163, 177);

3) Curvilinear methods for fitting non-linear models to a patient's response as needed to represent the data trend over time (Hayes, 1963, pp. 539-546);

4) Data transformations, such as log, logit, and so forth (Senn, 1997, pp. 112-114) to prepare the data for the analysis of an individual's response to treatment;

5) Generalizability theory to establish the dependability or “accuracy of generalizing from a patient's observed score on a test or other measure . . . to the average score that person would have received under all the possible conditions that the test user would be willing to accept” (Shavelson and Webb, 1991, p. 1);

6) A combination of the above to characterize in a progressive manner over time the course of each individual in a treatment;

7) A combination of these individually, fully characterized courses into categories, orders, ranks, by intervals or by ratios of individual patient responses characterized by the above or equivalent methods.

8) Analysis of variance, regression analysis, cluster analysis or other analyses known to anyone familiar with the art for the purpose of developing probabilities for long-term outcomes associated with each individual course (Hayes, 1963, 562-566; Senn, 1997, 95-109); and

9) Analysis of covariance, multivariate analysis, or other analyses known to anyone familiar with the art applied for each individual course in response to treatment conditioned by preexisting or emerging characteristics of individual patients to develop a model that takes into account preexisting or emergent characteristics of an individual in addition to the specific response to treatment. (Senn, 1997, 102-108).

Post-hoc analysis may be appropriate in extended clinical trials. If permutation or randomization tests do not allow the investigator to reject the strong null hypothesis, clinically significant differences may still be present within each treatment arm. This possibility can be explored by determining if the placebo or control arm of the study contains a significantly different distribution of subjects on the outcome variable between pre- and post-randomization. A significantly widened distribution of placebo treated subjects' scores comparing post-randomization outcome scores to pre-randomization outcome scores can occur with a reduced reliability in the outcome measures over time, a treatment or time effect on patients in the overall study not associated with the specific arms, or a placebo specific effect that may affect patients who would not respond in other treatment arms. Any of these conditions may have clinical significance and warrant further investigation.

A clinical trial is justified as externally valid for application in patient care by taking into account all the sources of error and bias that may affect the generalization from the trial conditions to patient care. Design and analysis are guided by the need for internal and external validity.

III. Applying the Evidence from Extended Clinical Trials:

A. In Patient Care.

To apply the model for evaluating individual patient responses in patient care, a practitioner complies with the assessment conditions needed to assure adequate reliability in outcome measures. Outcomes measures may be biased indicators, lose reliability, and compromise external validity of clinical trial evidence, when used outside double-blind conditions. Clinical trial investigators study outcome measures for reliability and bias during open followup of patients after the double-blind phase of the clinical trial to adapt the assessment plan for open use of outcome measures. To apply extended clinical trial evidence in patient care, a practitioner demonstrates sufficiently skilled use of outcome measures to reach test-retest reliability comparable to the reliability in the pre-trial study and complies with the assessment plan of the clinical trial. The practicing clinician uses or adapts to the individual patient situation the criteria of clinical and statistical significance from the trial to judge the response status of the patient and the probable level of confidence appropriate for the judgment of response status. The extended clinical trial provides confidence intervals for measurement of outcomes from treatment, and a model for assessing each patient's clinical course in relation to established medical and statistical criteria of significance. With these resources in hand, the clinician no longer depends on weakly supported clinical judgments or distribution-based probabilities from group comparisons to manage patients using clinical trial evidence.

For an example of an application of the research model generated by the methods of this invention, the present inventor assumes that, taking guidance from the method of the invention, regulatory authorities approve a drug for prescription with the indication that it is to be prescribed selectively to patients who benefit. Consequently, the managing physician uses one or more of the research tested means of assessment to ascertain patient response and selectively use the treatment in responders and to modify treatment in those without satisfactory response.

Within this method, the practicing physician during the course of management can employ as needed n-of-1 trials for individual patients to provide additional assessment of the benefits of treatment in the patient. A currently preferred embodiment of the present invention is directed to the use of this method of an n=1 clinical trial in a research CT to establish the conditions of use of an n=1 clinical trial in practice with individual patients as a means of demonstrating drug efficacy for patients who do not meet the CT standards of efficacy for an individual patient. Some patients in practice, or in the CT itself, may not meet the statistical standards of efficacy from application of the model to individual patients in the CT. An n-of-1 trial can be used to confirm or disconfirm the application of the model to this individual patient. Other patients, with the passing of time may deteriorate sufficiently that the CT confirmed model judges the probability of drug induced efficacy as less than the probability of no treatment effect. Again, an n-of-1 trial can be used to assess the prediction from these probabilities. The outcome of the n-of-1 trial can be fitted to the original model and probabilities of the treatment and placebo responses determined from the original model as described above. The comparative, probabilities or an odds ratio can inform the physician of the likelihood of benefit from treatment in this one patient. The n-of-1 trial can also be statistically analyzed within itself if the clinician prefers. The method of the invention allows a Bayesian analysis using the probabilities from the patient's treatment as priors, and the probabilities from the comparison of the n-of-1 trial outcomes to the model as posteriors, in addition to the frequentist probabilities used to interpret the trial in isolation. The method of the invention anticipates a CT confirmed model for the n-of-1 trial providing scientific grounding to interpretations of n-of-1 trials carried out in individual patient care. The clinician can use this method to test the efficacy of drug dosing changes, or beginning or ending dosing, prescribed in response to the clinical course of the individual patient.

For example, for either treatment successes or treatment failures the utility—the cost-benefit ratio—of maintaining or discontinuing treatment, or the odds ratio—even odds, or clinical judgment of the physician, may call for a confirmation of the clinician's evaluation and treatment intention supported by the clinical application of the research model. The clinician predicts a response to discontinuing drug, or to reinstating drug if the patient has already been discontinued. Using a blind sequence of periods of drug treatment and placebo treatment with assessments, the physician determines the regression slopes or means of treatment and placebo periods and applies the research model. Using the probabilities of the outcomes resembling the treatment ‘responders’ and placebo ‘non-responders’ in the research model, the physician takes his initial prediction as confirmed or disconfirmed with an associated probability. The clinician can use other methods as discussed above. Thus, the physician can confirm that an apparent responder or non-responder is or is not benefiting from the drug or device when support beyond the initial application of the research model is needed.

B. In Drug Development and Regulation.

In the randomized controlled trials that will support regulatory approval of a new drug, a pharmaceutical manufacturer may choose to use the method of the invention. The method of the invention better defines the appropriate place of the new therapy in patient care by the ability to individualize patient care decision making. Physician, family, and patient assessments are not mistaken as indicators of outcome when in fact they reflect the errors inherent in the methods of assessment or differences in response between patients. Patients can be maintained on a medication that will most probably benefit them or discontinued from a medication with no evidence of benefit opening opportunities for alternative forms of treatment. An expensive new treatment receives wider acceptance by hospital pharmacy committees and medical care funders as an approved treatment because the costs can be better controlled with access to research that uses the method of the invention.

A pharmaceutical company may recognize that a further investment to re-develop an already approved drug becomes economically advantageous in the competitive marketplace because the more selective use and its advantages to patients and medical care providers will have patent protection. The company may pursue a more precise regulatory approval available with the methods of the invention for its distinct advantages. Because of these advantages, the method of the invention can be used to develop a compound without patent protection because the developer could anticipate protection under the method of development. A drug with high costs for each treated case because a large number of persons would have to be treated to benefit a small number can be developed with the method of the invention to target the persons who are responders and to not treat the nonresponders, thus reducing the costs per patient benefited.

C. In Health Care Financing and Formulary Maintenance.

In one example, a new treatment shows equal efficacy to an earlier more expensive treatment for a medical condition. A small margin of additional mean benefit for the old treatment is disregarded by funders of medical care because of the high costs of the old treatment and the vague benefits. Applying the method of the invention to a clinical research study of the old treatment identifies significant additional efficacy for about 25% of patients and shows that a practitioner's brief use of the treatment to identify responders from non-responders adds only about 20% to the cost of treatment of each benefited patient. In view of the clinical significance of the benefits, and the ability to control the costs of each successfully treated patient, insurers, pharmacy committees, and other groups that control drug use, approve the old treatment used in conjunction with the method of the invention and methods applied to the new treatment.

In another example, an inexpensive medication effective in some patients with a given medical condition competes with an expensive medication more generally effective and free of adverse events. An organization responsible for funding medical care wishes to improve the overall quality of the care and to avoid unnecessary expenses. The organization agrees with the pharmaceutical manufacturer of the more expensive product that if they will develop a model for the effective, safe, and economical, use of the inexpensive and patented medications the organization will approve both for use and reimbursement. In a randomized controlled trial the manufacturer demonstrates how specific indicators of outcome can be followed in individual patients to identify, early in treatment, those who will reach maximum benefit on the inexpensive medication and those who will require the more expensive medication. This model becomes the standard controlling use of these medications in the medical care provided by, or reimbursed by, the funding organization. The scientifically highest quality medical care is provided, to patients at the lowest possible cost for such quality care.

D. In Electronic Medical Records.

Current plans for achieving widespread use of the electronic medical record within a decade recognize the easier access, reduced confusion interpreting reference standards that differ among providers, improved control over errors, and the ability to monitor medical care for response to community needs and overall quality of treatment. The electronic medical record develops out from the individual patients seen by the physician; it provides no support to making evidence-based medicine more immediately appropriate to and applicable in the care of the patient. The method of the invention offers this further step of a dynamic interaction of electronic medical records with CT evidence as a resource available to physician and patient to monitor medical treatments.

Interfacing the electronic medical record with an electronic record of the method of the invention, randomized controlled trials allow treatment decisions to take into account the specific probabilities for patient outcomes from different treatments and to monitor the progress of each patient in relation to the scientific evidence of individual patient courses and outcomes evidenced in the method of the invention randomized controlled research trials. Additional data about other patient outcomes can also be available to inform individual patient decision making and to monitor clinical practice experience in terms of research trial experience. The method of the invention integrates the aims of evidence-based medicine and electronic medical records to capitalize on these advantages by providing patient care that is scientifically and individually integrated.

An anxious or personally concerned patient who wishes to participate actively in her own health care explores the method of the invention modeled, randomized, controlled trials and traditional clinical trials relevant to her condition using Internet access to Medline and Medline plus. The better informed patient can select among treatments that seem most soundly justified to her and more easily engage in final decision making with the physician. The patient already in treatment can compare her progress with the research results in other trials. The patient better understands both her progress as a predictor of ultimate outcome using the method of the invention randomized controlled trials for her treatment and the place of her treatment among the options available for a person with her condition. Medical care is opened to the patient participating in the applications of scientific evidence in her care: the patient is more fully informed; the physician deals with a better educated patient without the burden of summarizing the relevant literature for the patient. Less mystified, more centrally involved, better able to reach sound preliminary scientifically based choices, the patient takes a greater interest in her own health.

E. In Pharmacy Practice.

Using the model of the method of invention, individual patient response data can become available in pharmacies to inform or control drug dispensing. Under this application physicians and pharmacists cannot, without active disregard of the approved standards of practice and method of implementation, alter the dosing and dosing schedules used in the CT, and thus violate the sound medical-scientific standards for drug use in patients. Examples of such protections of patients are a method for single dose packaging of a once-weekly dose of drug labeled by specific week and dispensed by a pharmacist after registering the patient using the patient's social security number or another unique identifier, and confidential code to access an electronic medical record of the patient's status interpreted by the model developed by the method of the invention. In this application, the patient's unique identifier can be recorded in a central registry maintained by the drug manufacturer or distributor. Pharmacists are required to consult this registry prior to dispensing the drug by prescription to avoid patients, by error or commission, receiving more than the dose or dosing confirmed as efficacious and safe in the CT.

IV. Illustrative Examples of the System and Method of the Invention:

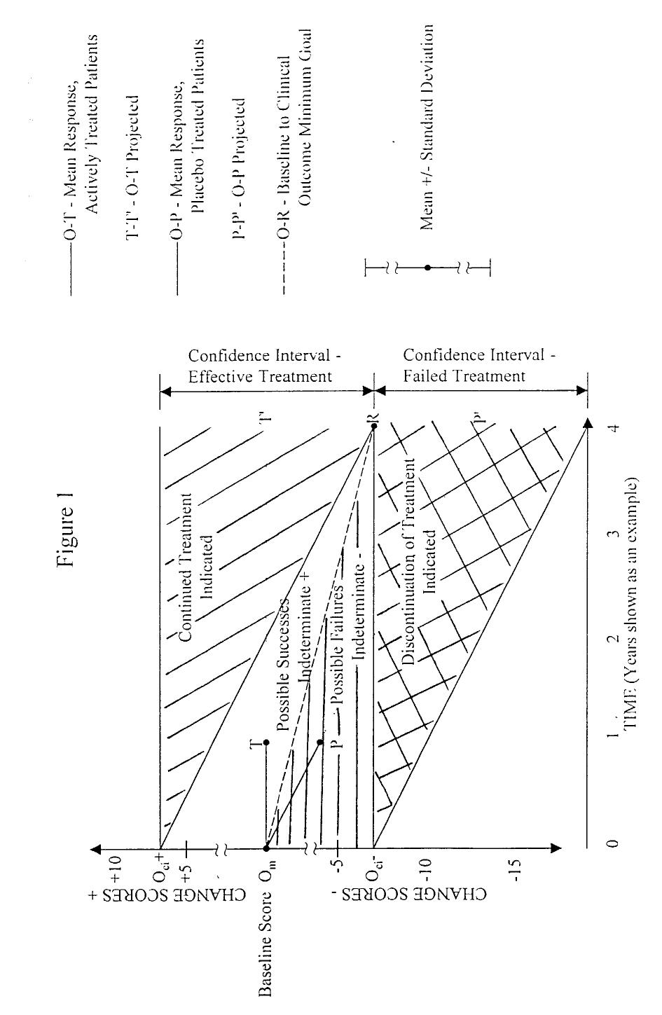

In accordance with the system and method of the invention, patients can be tested once only on multiple occasions or a single occasion, or in addition have only one of the triple assessments used for a data analysis, or tested more frequently or less frequently as needed to achieve reliability and validity sufficient to meet the intent of the CT and applications. The regression lines fitted to the data or other statistical summary of the data, or the data itself for each patient are fitted to a statistical or other model, such as the statistical model of FIG. 1, so that each pre-treatment or prior assessment(s) originate at a common point, in FIG. 1 point Oₘ. The data for the patients are used to calculate Confidence Intervals (CI) for the methods of assessment and levels of confidence are adopted using established methods. (See Becker and Markwell (2000) and others).

The individual regression lines, individual mean score, or other individual statistics are used to calculate mean regression lines or mean statistics, or other statistical summaries for data over time, for the drug treated and placebo treated groups, or among different treatments. These are shown as lines Oₘ−T and Oₘ−P in FIG. 1. (All similar references are to FIG. 1 unless stated otherwise). The points T and P are shown as an example at 1 year but can be of different time duration according to the design of the CT. The lines Oₘ−T and Oₘ−P are then projected to T′ and P′, or trends, means, or other summary statistics are projected taking into account the time course of individual patients, and the CIs under each of the conditions in the research are calculated and plotted. The time interval of the projection can vary according to the requirements of the CT and its intended applications. Based on acceptable probabilistic grounds, the regression lines or data of the research subjects are then ordered, ranked, scaled, or categorized, as appropriate (for example, as Continued treatment indicated, Indeterminate +, Indeterminate −, or Discontinuation of treatment indicated,) and the distributions of outcomes for each variable for the treatment and control-placebo groups determined. These analyses may be incorporated in other statistical treatments of the data set or may incorporate other statistical treatments by taking into account the effects of these analyses on the statistical conclusions that can be drawn. Prior to the trial the investigators will set statistical and clinical criteria of significance. The statistical criteria will fit the art, usually a p=0.05, to reject a null hypothesis. The clinical significance criteria will express the clinical aims for treatment acceptable to those familiar with the art. The clinical criteria can be applied to distinguish responders and non-responders in the trial. Then, the statistical criteria can segregate each group into probable or possible. For example, note that with a p=0.05 for the individual fitting the opposite condition of treatment in the research, the individual's clinical course falls outside the 95% confidence intervals of measurement for the mean comparison group clinical courses in FIG. 1. Or, the comparison can be made to the actual courses in the comparison group and the individual have only a 5% chance of her course occurring under comparison conditions.

### EXAMPLE 1

In one example, a sample of AD patients are randomly assigned blindly in a CT to receive an acetylcholinesterase inhibitor (“AchEI”) or placebo. The patient groups are assessed on the Alzheimer Disease Assessment Scale-Cognitive Sub-scale (ADAS-C) (Rosen et al., 1984), MMSE, and other behavioral outcome measures on three occasions, then begun on drug or placebo. Patients are treated for one year and assessed at three weekly intervals at 1, 3, 6, 9 and 12 months. For each outcome measure the assessments at three weekly intervals are plotted individually for each patient and a regression slope for the pre-treatment, 1,3,6,9, and 12 month three weekly averages, is calculated. The repeated assessments are used to calculate 95% confidence intervals. The regression slopes for the drug treated and placebo treated subjects are averaged separately to develop mean regression slopes for the two treatment groups. The confidence intervals are applied around these mean regression equations. The distributions of the outcomes are compared between drug treated and placebo groups to determine if the model identifies a sufficient excess of ‘responders’ in the treatment group compared to the placebo group, and a sufficient excess of ‘non-responders’ in the placebo group compared to the treatment group to reach statistical significance. If the distributions differ with a statistical significance at p=0.05 or less probable occurrence by chance, then the model is accepted as a means of identifying drug responders and non-responders using probability. The CT outcomes for each patient course at each assessment are used to provide a predictive model for interpreting in medical practice an individual's course in relation to the distributions of individual outcomes in the CT.

### EXAMPLE 2

An analysis of a sample from an actual study of AD patient treatment is given below in order to provide a more complete illustration. In analysis of follow-up at one year of the treated group from a CT, although patient responses are less different from placebo than at the six-month end of the original research, they remain statistically significantly different from the placebo group courses projected from the original research. This one-year data set can be used to assign probabilities for patient outcomes in clinical patient care. At two years the distributions of research patients in followup are not significantly statistically different; it would not be valid to assign probabilities to patients whose status changes over time based on this two year data and to interpret these probabilities as reliable and valid reflections of the probability of efficacy and safety of the treatment. However, a patient who remains unchanged in status (within the projected original confidence intervals from the CT or within the confidence intervals and course projected from the period of treatment within the duration of the CT and shown as the course of a responder according to the CT) can still be validly assigned probabilities from the original model if the assumptions of stable placebo response over time are accepted.

The probabilities of patient courses that change over time, lead to distributions that are no longer statistically significantly different from placebo or comparison treatments, and thus do not fit the assumptions for extension of the original analysis, can be reassessed by randomization of groups to drug or placebo in a new clinical trial or by n-of-1 trials of sufficient patients with the same duration of drug exposure to obtain statistical evidence for differences either in relation to the original placebo group distribution or in relation to drug and placebo treated periods in the n-of-1 trials. From the example cited above, after two years when patient changes during treatment cause many patients to no longer fit the original research model, a series of n-of-1 trials of patients can show whether treatment is effective for individuals compared with periods of placebo.

N-of-1 trials suffer from requiring a number of sequences of placebo and drug and from low power in a statistical analysis. The method of the invention overcomes these difficulties by allowing sometimes only one blind, randomly assigned, placebo comparison to drug to evidence efficacy or the lack, or safety or the lack, because of the power added by Generalizability theory to within subject comparisons. Criteria of success and failure can be whether the subject's clinical course remains within or falls outside the confidence intervals from the original research model; or remaining within or falling outside the predictions from Generalizability theory from the course of the patient to date. Thus, n-of-1 trials become more practical since they can be of shorter duration and increased power by incorporation within the model developed in a CT using the method of invention.

### EXAMPLE 3

In another example, a favorable profile of blood glucose induced by a drug may prove to have different long-term outcomes predicted for a patient who diets, exercises and loses weight while the same initial profile of response will deteriorate and have an increased risk of secondary consequences of diabetes mellitus in a patient who does not observe dietary restrictions, exercise, and lose weight. Or, in two patients who differ only in not losing weight even though they diet and exercise, the same degree of initial research control of blood glucose may have different long-term consequences in followup because initially one patient was 5% below optimal body weight and the other patient was 40% above optimal body weight.

### EXAMPLE 4

This model is applied in patient care using the same methods of assessment and statistical summary of individual patient experience used in the CT. For example, the physician, using a statistical model displayed in graphical form, such as in FIG. 1, or a statistical model or an arithmetic or mathematical model used in the CT(s), decides on continued treatment or discontinuation of treatment for individual patients based on a reliable plot or expression of a patient change score (perpendicular axis), time course, or other assessment, taking into account the time of acquisition (horizontal axis). The physician to assure reliability may use a sequential plot or statistical summary, such as the regression slope, mean, or other scientifically, medically and statistically sound comparisons of the individual patient to the CT model. The physician may predict outcomes using the probability of outcomes in the CT for patients with the same score as the immediate patient in clinical care, or trend of scores over the course of treatment, or for the group of patients within the CI of the score or trend or for all patients in the same category (in our example responder, indeterminate +, or for the mean of subjects in the research that fall within a CI generated from the treated patient's course, and so forth). The most current category of the patient, the probabilistic prediction of the immediate or ultimate benefit or failure from continued treatment, tested in the CT model or statistical method, determines the subsequent care of the patient. In this manner over time, and in awareness of the contributions of the error introduced by the methods of assessment, the physician categorizes patients as treatment responders or non-responders, or as in the future probable responders or non-responders, by inferentially applying the medically and scientifically sound methods of individual patient assessment tested and confirmed in the CT. The treatment is individualized to each patient appropriate to the level of reliable and valid assessment for that patient at specific times after initiating treatment.

### EXAMPLE 5

In one example of an application of the research model generated by the methods of this invention, we assume that, taking guidance from the method of the invention, regulatory authorities approve a drug for prescription with the indication that it is to be prescribed selectively to patients who benefit. Consequently, the managing physician uses one or more of the research tested means of assessment to ascertain patient response and selectively use the drug or device in responders. The practicing physician can use repeated single or averaged assessments to determine the patient's place in the data set generated by the research model. Based on patient A maintaining performance greater than the mean of the treated group, the physician continues treatment since the indication is that the patient resembles the group of patients who were ‘responders’ in the research. Another patient B who declines more than the average of the placebo group most likely is not benefiting and is discontinued as a treatment failure. For patients C, D, and so forth, with outcomes between these extremes, the physician continues or changes or discontinues treatment according to the probability in the odds ratio between the patient resembling the treated or placebo patients in the research.

### EXAMPLE 6

An important example of the application of the model developed by the method of the invention occurs when the period of clinical treatment goes beyond the period of the original double-blind CT. A patient may show for 18 months a response greater than the average of the drug treated research group. At this point the patient may begin to decline. The decline may be less than, parallel to, or greater than, the mean regression equation of the placebo group in the research. The patient may still generate an odds ratio greater than 1 in the original model because of the 18 month response. The physician can apply the original model projected beyond the duration of the original study to generate probabilities for the patient, can move the origin of the original model in time to the present and apply the model to the current data, and can carry out an n-of-1 trial. The original model may identify the patient as currently a responder, the model with origin moved into the present may identify the current trend as probably that of a non-responder. The physician hypothesizes the patient is no longer responding and increases the drug dose without effect. The physician then carries out an n-of-1 trial and determines that the patient response does not differ on or off drug. The physician can rationally discontinue this drug therapy or seek changes in the patient that alter the response. Accordingly, the method of invention provides improved scientific and statistical grounds for monitoring the long-term management of patients in treatment.

### EXAMPLE 7

The researchers, investigating a new potential therapeutic, conduct CT(s) in which the outcome measures are used with each subject on sufficient occasions to develop statistically and clinically satisfactory CIs for the measures used in individual patients. To develop the drug for use in individual patients the researchers apply the methods described herein. They use the method of calculating confidence intervals (CI) for the methods of assessment both prior to and after the treatments to determine whether there are interactions between the outcome errors and treatments. Drawing on standard means of assessing the outcome of treatment for the condition addressed in the patients, the investigators carry out, blind to the drug-placebo status of the research subjects, weekly assessments of the patients on three consecutive weeks, prior to randomization to drug or placebo and then at two monthly intervals over the course of the study. They plot regression lines or means as appropriate for the individual patients using the assessments or means of assessments according to the need to restrict the breadth of the CIs. They then calculate a mean or a mean slope for the drug treated patients and another for the placebo treated patients. They apply the CIs and determine the numbers of patients, for drug and placebo treated groups, improved, indeterminate +, indeterminate −, and unimproved, on the model of FIG. 1. They statistically determine that the drug treated group distribution favors improved outcome at a p=0.05 and the model application of the drug treatment is accepted by the regulatory authorities for prescription use. The model shows a 95% CI for the major outcome measure of +/−4.00 for comparison of a pair of single assessments, and +/−2.50 for comparison of a pair of means from three averaged assessments. Assessments could be blood pressures, blood chemicals, imaging, questionnaire ratings, and so forth to include potentially all outcome measures in modern medicine. The model can express secular or progressive changes characteristic of the disease course. For example, the model shows a mean decline in the outcome measure of 4 per year for the placebo group and 0 per year for the drug treated group.

### EXAMPLE 8

The method of the invention can be illustrated by re-analysis of a sample of patients from the studies of Becker et al., 1996 and 1998, which are hereby incorporated by reference as part of the present disclosure. The original studies were analyzed using current CT methods and led to the conclusion that the results could not be selectively applied to the majority of individual patients. (Becker and Markwell, 2000). The re-analysis using the methods of invention illustrates how this method develops a model that can be applied to individual patients in medical care.

On a random sample of 15 patients who received placebo for 3 or 6 months and a random sample of 15 patients who received drug for 3 to 6 months, and 15 patients who were treated with drug for one to three or more years (each drawn from the subject pool in Becker et al., 1996, 1998, the present inventor uses the CIs obtained for the larger group and reported by Becker and Markwell (2000)). In Becker and Markwell (2000) the methods for calculating confidence intervals for test-retest comparisons of outcome measures were applied to the two study groups of patients from whom these illustrative samples are taken. The three and six month mean differences from baseline are used to plot the course of each patient and the CIs drawn on the plot following the method illustrated in FIG. 1. The mean slope of the treated and placebo patients is projected and found to predict zero points decline for active treatment per year and about 4 points decline for placebo treatment per year. Using the division of outcome by the odds ratio=1 for probability of a treatment effect versus probability of a placebo effect (line Oₘ−R in FIG. 1) the following distributions of outcomes are found:

| | Probable responder | Probable non-responder |
|---|---|---|
| Drug treated | | |
| 3 or 6 months | 13 | 2 |
| 1 year | 8 | 7 |
| 2 years | 4 | 9 |
| 3 years | 4 | 6 |
| Placebo treated | | |
| 3 or 6 months | 1 | 14 |

The distribution of the patients was statistically tested using Fishers Exact Probability Test. (Siegel, 1956, p. 96-104). The 3 or 6 months distributions are significantly different with p less than 0.05, which would lead to the interpretation that the drug is effective in patients who respond. Since the rate of decline fits the body of published long-term decline data of about 4 points per year in untreated patients, the unblinded followup can be compared to the placebo distribution projected over the period of followup. The difference remains significant at one year and loses significance at years two and three. Unlike the generalization that could be drawn in practice from the original analyses using current CT methods—that drug efficacy is not qualified by time—the re-analysis 1) supports drug efficacy only for one year and 2) demonstrates that this efficacy receives probabilistic support from the CT only for patients who can be shown to be responders in the terms of the model.

During the period of treatment of this sample, some patients were blindly assigned to drug or placebo for six months and rated on three consecutive weeks prior to assignment and then at two, four and six months after assignment. There are also two, four, and six month ratings for the 6 months prior and subsequent to this blind rating period. This allows us to illustrate the n-of-1 trial used in the research model and as it could be used in the applications described below.

Patient 4 declined steadily over 3 years with an Odds Ratio that favored a response that matched the placebo group. Over the 6 months prior to three years the patient declined by 1 point, during a blind placebo treatment for 6 months at three years (comparable to an n-of-1 trial) the patient declined by 1 point and in the subsequent 6 months on drug the patient declined by 1.6 points. The model of the invention predicted this patient to be a nonresponder; an n-of-1 trial confirms the patient as a nonresponder because the drug-placebo difference in the n-of-1 trial does not match the trial model nor does it reach statistical probability in a test of means or slopes.

Patient 5 declined steadily, but over 3 years the patient's response favored the patient being a drug responder. In an n-of-1 trial under the conditions described above the patient showed a one point decline during two drug treatment periods and a 1.6 point decline during the placebo treatment period. These differences are not statistically different and in the model favor no evidence of a drug effect. Both patients 4 and 5 can be discontinued and another treatment considered.

Patient 15 declined at an average of 5 points per year for 3 years strongly suggesting patient 15 was a treatment failure. In the n-of-1 trial, patient 15 declined by 1.6 points in each of the two drug treated six month periods but by 3.5 points in the six month placebo period. Fitted to the model this does not confirm the initial interpretation that patient 15 is a treatment nonresponder and the 3.8 point projected per year difference in slopes indicates to the clinician that the drug has an effect that is benefiting the patient.

These examples of n-of-1 trials illustrate how this methodology can be used to obtain double-blind longitudinal data to expand the research data base of the model and thus supplement the original drug efficacy evidence. It is probable that patient 15 would have been discontinued as a treatment failure if a clinician had only the data of the original research analysis (Becker et al., 1996, 1998) and that patients 4 and 5 may have remained on treatment—a treatment that did not benefit them and an unnecessary medical care expenditure. If additional n-of-1 trials are conducted on the patients at 2 and 3 years treatment, a statistically significant altered model for 2 and 3 years might emerge or loss of efficacy might be confirmed.

Improved Support of the Practitioner.

### EXAMPLE 9

In another example, the method of invention can be employed in reinterpretation of currently analyzed trials as hereinafter described. A physician managing an Alzheimer's disease patient finds no change in Mini-Mental State Examination scores justification for switching drugs since the advertising for the product—based on current methods of CTs and their analysis—specifies a “4-point improvement” over placebo treated patients. If the physician has analysis of the invention available, he would know that this patient's six-month score predicts with a high probability one of the most successful available longer term treatment outcomes and that he could confirm this prediction using the model generated by the method of the invention to interpret an n-of-1 trial.

### EXAMPLE 10

In another example, a physician treating a depressed patient finds the patient not “fully” recovered after one month. The physician knows that customary practice and research evidence require at least 6 to 12 weeks of treatment to minimize the chances for relapse in this first depression for this patient. Under pressure from the patient, the physician agrees to try a new drug. If the physician had available the individual clinical courses of patients treated in the randomized clinical trials as would be developed by the method of the invention, the physician would realize that the patient's progress predicts with high probability full and lasting recovery after three months of drug treatment.

### EXAMPLE 11

In CTs, the method of the invention for a newly approved disease-modifying antirheumatic drug provides a range of patient courses and for each course the probability of ultimate improvement of rheumatoid arthritis, given the current dose and duration of treatment at that dose. The model also provides the probability of maintaining improvement at different levels of dosage reduction once maximum benefit has been reached. Using the model developed from the method of invention in CTs as a reference, an electronic medical record system routinely queries a patient and enters these assessments with the laboratory studies and clinical findings to monitor the current treatment in terms of whether it offers the patient the highest probability of optimal outcome long-term. The physician's assessments, abbreviated by the demands of a busy practice, are supplemented by the patient's self reports and the automatic analysis using the model developed by the method of the invention.

### EXAMPLE 12

An anxious or personally concerned patient who wishes to participate actively in her own health care explores the model of the invention derived from modified, randomized, controlled trials and traditional clinical trials relevant to her condition using Internet access to Medline™ and Medline plus™. The better informed patient can select among treatments that seem most soundly justified to her and more easily engage in final decision making with the physician. The patient already in treatment can compare her progress with the research results in other trials. The patient better understands both her progress as a predictor of ultimate outcome using the method of the invention randomized controlled trials for her treatment and the place of her treatment among the options available for a person with her condition. Medical care is opened to the patient participating in the applications of scientific evidence in her care; the patient is more fully informed; and the physician deals with a better educated patient without the burden of summarizing the relevant literature for the patient. Less mystified, more centrally involved, better able to reach sound preliminary scientifically based choices, the patient takes a greater interest in her own health.

### EXAMPLE 13

A new treatment shows equal efficacy to an earlier more expensive treatment for a medical condition. A small margin of additional mean benefit for the old treatment is disregarded by funders of medical care because of the high costs of the old treatment and the vague benefits. The method of the invention applied to a clinical research study of the old treatment identifies significant additional efficacy for about 25% of patients and shows that a brief trial to identify responders from non-responders adds only about 20% to the cost of treatment of each benefited patient. In view of the clinical significance of the benefits, and the ability to control the costs of each successfully treated patient, insurers, pharmacy committees, and other groups that control drug use, approve the old treatment used in conjunction in accordance with the invention.

Selective Use of More Economical Methods of Treatment in Those Individual Patients Who Show No Additional Benefits from More Expensive Methods of Treatment.

### EXAMPLE 14

An inexpensive medication effective in some patients with a given medical condition competes with an expensive medication more generally effective and free of adverse events. An organization responsible for funding medical care wishes to improve the overall quality of the care and to avoid unnecessary expense. The organization agrees with the pharmaceutical manufacturer of the more expensive product that if they will develop a method of invention model for the effective, safe, and economical, use of the inexpensive and patented medications, the organization will approve both for use and reimbursement. In a randomized controlled trial the manufacturer demonstrates how specific indicators of outcome can be followed in individual patients to identify, early in treatment, those who will reach maximum benefit on the inexpensive medication and those who will require the more expensive medication. This model becomes the standard controlling use of these medications in the medical care provided by, or reimbursed by, the funding organization. The scientifically highest quality medical care is provided to patients at the lowest possible cost for such quality care.

### EXAMPLE 15

Current plans for achieving widespread use of the electronic medical record within a decade recognize the easier access, reduced confusion interpreting reference standards that differ among providers, improved control over errors, and the ability, to monitor medical care for response to community needs and overall quality of treatment. The electronic medical record develops out from the individual patients seen by the physician; it provides no support to making evidence-based medicine more immediately appropriate to and applicable in the care of the patient. The method of inventions offers this further step.

Interfacing the electronic medical record with an electronic record of randomized controlled trials in accordance with the invention allows treatment decisions to take into account the specific probabilities for patient outcomes from different treatments and to monitor the progress of each patient in relation to the scientific evidence of individual patient courses and outcomes evidenced in the randomized controlled research trials of the invention. Additional data about other patient outcomes can also be made available to inform individual patient decision making and to monitor clinical practice experience in terms of research trial experience. The method of the invention integrates the aims of evidence-based medicine and electronic medical records to capitalize on these advantages by providing patient care that is scientifically and individually integrated.

### EXAMPLE 16

Using the method of the invention, individual patient response data can become available in pharmacies to inform or control drug dispensing by prescription. Under this application, physicians and pharmacists cannot, without active disregard of the approved standards of practice and method of implementation, alter the dosing and dosing schedules used in the CT and thus violate the sound medical-scientific standards for drug use in patients. Examples of such protections of patients are a method for single dose packaging of a once-weekly dose of drug labeled by specific week and dispensed by a pharmacist after registering the patient using the patient's social security number or another unique identifier and confidential code to access an electronic medical record of the patient's status interpreted by the model developed by the method of invention. In this application, the patient's unique identifier can be recorded in a central registry maintained by the drug manufacturer or distributor. Pharmacists are required to consult this registry prior to dispensing the drug by prescription to avoid patients, by error or commission, receiving more than the dose or dosing confirmed as efficacious and safe in the CT.

### EXAMPLE 17

Dr. A instructs her patient Mr. B to monitor his blood glucose and blood pressure regularly as part of the management of Mr. B's Diabetes Mellitus Type II and Essential Hypertension. Dr. A recommends to Mr. B dietary restrictions, an exercise program, goals for weight loss, and prescribes an oral hypoglycemic medication and an antihypertensive medication. Mr. B uses monitors for blood glucose and blood pressure designed for the home. These monitors can be connected by phone to an Internet site. Dr. A maintains an electronic medical record for each patient. The Internet site integrates the reports of Mr. B's self monitoring into the medical record and analyses the findings in relation to Mr. B's clinical course and in relation to comparable patients in the randomized controlled trials that determined the safety and efficacy of the medications.

To become available for prescription, each prospective medication must demonstrate that it is effective and safe in randomized controlled trials. The research clinical trials that supported the regulatory approval of the drugs Mr. B receives incorporated the methods of the invention. This made available individualized assessments of each research patient's experiences. These individual records of patient drug responses and outcome from treatment are available to the Internet site that maintains Dr. A's electronic medical records. Consequently Mr. B's responses over the time of treatment can be interpreted in relation to the patient experiences individually evaluated and validated in the clinical research. This analysis uses the model of the present invention each time Mr. B submits new findings and provides him and his doctor specific interpretations based on the actual experience of earlier patients and explicit probabilities for most likely outcome from continued treatment. Dr. A does not just use the best evidenced medication to treat her patients; she and her patients collaboratively monitor closely the effectiveness and safety of each dose of medication because they can compare Mr. B's responses to the specific patients evaluated under research conditions in randomized controlled clinical trials of the drugs.

### EXAMPLE 18

Shortly after beginning a new oral antidiabetic medication a single high blood glucose measurement worries Mr. B and he submits the finding to his electronic medical record. The Internet site recognizes this reading as within the range of values found after the same duration of treatment in the research study with successfully treated patients and reassures Mr. B. If this same value had occurred months later the value would evoke a warning that blood glucose control is not adequate. The analytic program incorporates in its response to the patient and doctor the probable progression to close control found in the clinical research with the drug.

Mr. B submits a blood pressure reading he recognizes as unusual. He confirms the reading twice. The site recognizes the initial reading as within the error range for single readings but finds the mean of the three readings a significant deviation. The site reassures Mr. B that this deviation does not require immediate action on his part. The site notifies Dr. A who evaluates the finding and calls the patient.

### EXAMPLE 19

After six-weeks treatment for his hypertension at an office visit Mr. B points out that his neighbor “got back to normal blood pressure already and I'm not there even though we started together.” Dr. A sees the progress but wonders if something more should be done for Mr. B. She considers changing his medication. Before doing so she compares his course to the course of research patients. The comparison indicates that he has an 80% —4 to 1 odds—that he will be at a blood pressure of 140/85 within 3 months and improve further for an additional 3 months. Since he has not demonstrated interfering adverse effects from his current medication, she shares the information with him and they decide to remain on the current medication.

### EXAMPLE 20

Some months later Mr. B forwards a high blood glucose reading. The Internet site asks if he has strayed from his diet or skipped his exercise that day. Mr. B reviews his last meal and notes that he conveniently overlooked the glycemic index of a treat. He is reminded immediately of the cause of his difficulty and becomes more vigilant in his self care.

With a monitor available that continuously samples for a patient's blood glucose a drug company uses the method of this invention to record the daily blood glucose profiles of each research patient. By subtracting the area under the curve of a normal range of blood glucose from the area under the curve of each patient's blood glucose profile the research provides both individual profiles of response and the incremental accrual of, and total daily, excess glucose exposure over time within those profiles. The research goes on to key these exposures to surrogate markers of complications by long-term follow-up of research patients and from other research sources. The data from a monitor worn by Mr. B is entered into his electronic medical record over the Internet and interpreted with the method of the invention. Dr. A then can evaluate Mr. B against this data base and achieve closer control of blood glucose with the new medication. The extent of the problem of inadequate control of blood glucose is available each day and does not have to wait for tests for glycosolated hemoglobin. Mr. B, by viewing the progression of daily blood glucose plots over months of treatment, becomes reinforced in his adherence to the management regimen by the evidence of progress and the immediate increased probabilities for worsened outcome when poor glucose control occurs.

Other Patient Examples:

Physicians prescribe the medication to individual AD diagnosed patients after either 1, 3 or more consecutive MMSE assessments. The following are different physicians' experiences with patients and their use of the model. For practical application the model is available in Graphical form, such as FIG. 1, or in a computerized presentation where the physician enters the patient's scores and dates of acquisition and the computer or calculator automatically displays the patient's status. The application references the illustration developed as a re-interpretation of the already published AD studies:

Patient A at 3 months has a single MMSE of 25 compared to a single pre-treatment MMSE of 21. This is 1/4 point above the upper limit of the single assessment CI around the placebo mean regression line at 3 months. A is a responder and treatment is continued.

Patient B at 3 months has an average score of 15 on the MMSE (scores on weeks 10, 11, 12 are 15, 13, 17) compared to an average immediately before starting the medication of 18 (three consecutive scores of 20, 19, 15). This score chance falls below the lower 95% CI for mean scores of the placebo group characterizing the patient as a probable treatment failure. The physician decides to follow the patient for another month, finds the patient continues on the same rate of decline with a mean score of 14. The physician blindly discontinues treatment for a month by arranging for the pharmacist to provide a placebo. The patient remains at 14 for this month. They accept the patient as a treatment failure. The physician turns to another treatment.

Patient C has already had two years of treatment with another drug for AD but has declined from 26 on the MMSE to 19. Regarded as a failure by the treating physician the patient has another form of treatment with a decline to 16 in 6 months. This too is regarded as a failure and the model from the method of the invention is used to determine whether this patient can benefit from treatment. After starting the newly approved drug, the patient at 12 months has a single MMSE of 12 compared to a single pre-treatment MMSE of 16. This is 4 points decline in a year, the expected placebo decline in the CT model, a rate of decline that projects that, if sustained, the patient will have a higher probability of being a treatment failure than a treatment success. The physician arranges with the pharmacist to dispense, physician and patient blind to the condition, drug or placebo to the patient over the next year alternating the condition randomly every 3 months. The physician selects this course because in the research model developed by the method of the invention (see FIG. 1) even some patients that were apparent failures were benefiting from drug. The physician blindly assesses the patient every three months and then plots the course using the model. At the end of the two years the patient has an MMSE score of 10. The physician has predicted the patient will be a clear treatment failure from the 4 point initial decline at 1 year; in spite of 6 months on placebo the two year assessment does not confirm that prediction. In two of the three month drug treated periods in the second year the patient declines 0 and 1 points, and in two three month placebo periods, the patient declines 2 and 3 points. The data fitted to the model do not support his prediction that drug treatment will be ineffective. The patient is continued on medication with the prediction of no more than 2 points per year decline because this is the confidence interval from the patient's course fitted to a 1 point per year projected decline from the n-of-1 trial. If the decline does not exceed a rate of 2 points per year, the physician receives support from the model that the patient receives some benefit from the drug.

Patient D has been treated using the model from the illustrated CT(s) for 2 years. At a baseline mean of three MMSE ratings she scored 25. At 1 year she scored 26 as a mean of three ratings. At two years she scored 21 as a mean of three ratings. Her physician worries that the rate of decline between year 1 and 2 reflects a loss of drug effect. The patient was a responder at year 1, but is now in the indeterminate +group. The physician follows the patient with mean ratings every 3 months for the next year. The patient has regression lines fitted to the data by a computer program available to the physician in a hand-held or desk-top computer. Using all assessments, the equation shows at 3 years an MMSE score change of −4 placing the patient strongly in the responder group in the model. The physician worries that this is an artifact of the patient's initial favorable response and produces a regression equation without using any data obtained prior to the year 1 assessments. The equation shows a MMSE 3 year score of −7 which is −1 CI below the mean treated group model projection from the CT, but +2 CIs above the projected placebo decline. This reassures the physician that benefit is probably ongoing. If this second regression equation had shown over time the difference in the patient score from the projected mean placebo regression projection progressively growing smaller, the physician would be supported by the model to change the patient's status from responder (at 1 year) to probable non-responder with continued treatment. This would justify the physician reevaluating the patient's current treatment: using an n-of-1 trial; changing treatment; and so forth.

Patient E has an initial single assessment of 18, at three months 17, at 6 months 15, at 9 months 14. The patient shows a trend that will clearly indicate probable treatment failure during the 2^{nd} year if the trend is maintained. Since the patient will be severely cognitively impaired if the trend continues, the physician considers the patient a failure on this treatment and turns to other treatments that have shown promise in AD.

As may be recognized by those of ordinary skill in the pertinent art based on the teachings herein, numerous changes and modifications may be made to the above-described and other embodiments of the present invention without departing from its scope as defined in the appended claims. For example, the system and method of the invention and any of its applications can be embodied in a computer software program, a published set of directions, flow charts, worksheets, instructions, guidelines, technical training, skill training, or other forms, and result in publications in articles and books, audiotapes, CD recordings, or other forms. Accordingly, this detailed description of preferred embodiments is to be taken in an illustrative as opposed to a limiting sense.

I claim:

1. A method of conducting a CT that enables assessment of an individual patient's response to a drug or other medical procedure used to treat a condition of the patient, the method comprising the following steps:
identifying the aims of the CT and the anticipated applications of the CT in patient care;
identifying proposed outcome measures of each patient's medical condition, and determining whether the proposed outcome measures have sufficient reliability to meet the aims of the CT and the anticipated applications of the CT in patient care;
conducting a reliability study of at least one outcome measure to be used in the CT and determining the error of measurement of the at least one outcome measure based thereon;
developing an assessment plan for the CT by selecting the frequency and form of measurement of each patient's medical condition based on an error of measurement offering sufficient reliability to meet the aims of the CT;
identifying criteria of clinical significance for use in the CT and in applications of the CT in patient care;
selecting criteria of statistical significance to set the level of chance occurrence for use in interpreting comparisons in the CT;
assessing a plurality of patients in the CT in accordance with the assessment plan;
comparing each patient's clinical course to the criteria of clinical significance, and determining whether the patient's condition is improving or not based thereon;
estimating the probability that the drug or other medical procedure is necessary for improvement of an individual patient's condition by comparing the chance occurrence of each individual patient's clinical course among active and placebo treated patients in the CT; and
determining based on at least one long-term outcome of the CT whether the measured improvement will result in a long-term favorable outcome for the individual patient.

2. A method as defined in claim 1, wherein the reliability study is a test-retest reliability study.

3. A method as defined in claim 1, wherein the step of determining the error of measurement includes determining the error of measurement of a single administration of an outcome measure and the error of measurement for multiple administrations of an outcome measure summarized as a descriptive summary statistic.

4. A method as defined in claim 1, wherein each patient's clinical course is characterized by the outcome measures carried out in compliance with the assessment plan.

5. A method as defined in claim 1, wherein the step of comparing each patient's clinical course to the criteria of clinical significance includes determining whether each patient meets the criteria of clinical significance and identifying each patient as a responder or not based thereon.

6. A method as defined in claim 1, further comprising the steps of assessing an individual patient's response to a drug or other medical procedure used to treat a condition of the patient by:
treating the patient in accordance with the assessment plan of the CT;
confirming that the error of measurement for the at least one outcome measure applied to the individual patient does not exceed the error of measurement for the corresponding outcome measure used in the CT;
comparing the patient's clinical course to the criteria of clinical significance from the CT, and determining whether the patient's condition is improving or not based thereon;
applying the criteria of statistical significance from the CT to estimate the probability that the drug or other medical procedure is necessary for improvement of the individual patient's condition; and
determining based on at least one long-term outcome of the CT whether the measured improvement will result in a long-term favorable outcome for the individual patient.

7. A method as defined in claim 1, wherein the assessment plan from the CT includes information concerning: (i) how frequently outcome measures are administered to patients; (ii) how multiple administrations avoid carryover effects; and (iii) which single measure or descriptive summarizing statistic for multiple administrations is used in data analysis to control error of measurement in a test of hypotheses in the CT.

8. A method as defined in claim 1, further comprising the step of selecting a single measure or a scalar summary statistic that summarizes multiple measures taken in relation to each other at or near a point in time and using the selected measure or scalar summary statistic to describe the patient's clinical course as a clinically significant response or non-response to the treatment received.

9. A method as defined in claim 8, wherein a confidence interval of measurement is used to judge the patient's clinical course in relation to criteria of clinical significance.

10. A method as defined in claim 1, wherein the step of estimating the probability that the drug or other medical procedure is necessary for improvement of the patient's condition includes at least one of the following comparisons: (i) the probability that the treated patient's course would occur under comparison or placebo conditions; (ii) whether a confidence interval of the treated patient's course overlaps or does not overlap a mean of courses within an actively treated or placebo treated group in the CT, (iii) an odds ratio of the cumulative frequency of the treated patient's course among actively treated patients divided by the cumulative frequency among comparison or placebo treated patients; and (iv) an exact probability comparing the treated patient to active and placebo treatment determined by a randomization test.

11. A method as defined in claim 1, wherein the step of estimating the probability that the drug or other medical procedure is necessary for improvement of the patient's condition includes calculating an odds ratio for each of a plurality of clinical courses occurring under treatment and placebo conditions.

12. A method as defined in claim 11, wherein the odds ratio includes the probability that a surrogate outcome indicates a treatment effect will result in a long-term health benefit.

13. A method as defined in claim 1, further comprising the step of applying the criteria of statistical significance to perform at least one of (i) determining whether an individual patient is a responder or not; (ii) establishing the probability that an individual patient's clinical course could occur under placebo or under treatment conditions; (iii) statistically supporting the internal validity of the CT; (iv) selecting confidence intervals; and (v) distinguishing as different two or more clinical courses.

14. A method as defined in claim 1, wherein the step of determining whether an individual patient's condition is improving or not includes using n-of-1 trials to confirm whether the patient is meeting criteria of clinical or statistical significance, or is experiencing a clinically significant or statistically significant effect of treatment compared with placebo.

15. A method as defined in claim 1 further comprising the step of providing confidence intervals for measurement of outcomes from treatment, and using the confidence intervals to test for treatment and placebo effects in n-of-1 trials.

16. A method as defined in claim 1, wherein the step of determining whether the measured improvement will result in a long-term favorable outcome for the patient includes generating probabilities for long-term outcomes specific to distinct clinical responses.

17. A method as defined in claim 16, wherein the distinct clinical responses include individual courses, and course intervals bounded by confidence intervals of measurement.

18. A method as defined in claim 17, wherein the differences among courses are measured by surrogate outcome variables with confidence intervals of measurement derived from the error of measurement.

19. A method as defined in claim 1, further comprising the step of providing confidence intervals for measurement of outcomes from treatment, and a model for a practicing physician to use to assess each patient's clinical course in relation to established clinical and statistical criteria of significance and individual patient courses in the CT.
